(19) United States
(12) Patent Application Publication
(10) Pub. No.: US 2024/0342017 A1
(43) Pub. Date: Oct. 17, 2024

(54) Modified Boxer Brief Underwear for Menstrual Cycles

(71) Applicant: Taylor Michelle Emery, Christiansburg, VA (US)

(72) Inventor: Taylor Michelle Emery, Christiansburg, VA (US)

(21) Appl. No.: 18/301,217

(22) Filed: Apr. 15, 2023

(51) Int. Cl. A61F 13/496

(52) U.S. Cl. CPC A61F 13/496

(57) ABSTRACT

A method for using boxer briefs to accommodate for menstrual cycles. An extra piece of fabric that is 100% cotton, for the purpose of application of pads with wings and panty liners, is stitched longitudinally on both ends within the crotch area. Due to the nature of the 100% cotton material, pads stick easily and are held in place during use by parallel stitching located up and through the inside of the front of the boxer briefs until the waistband.

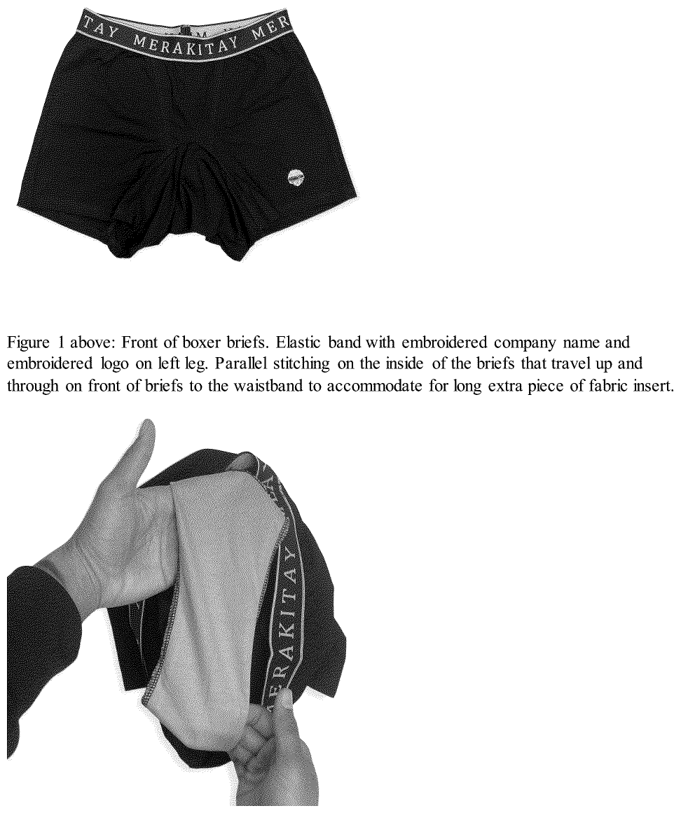

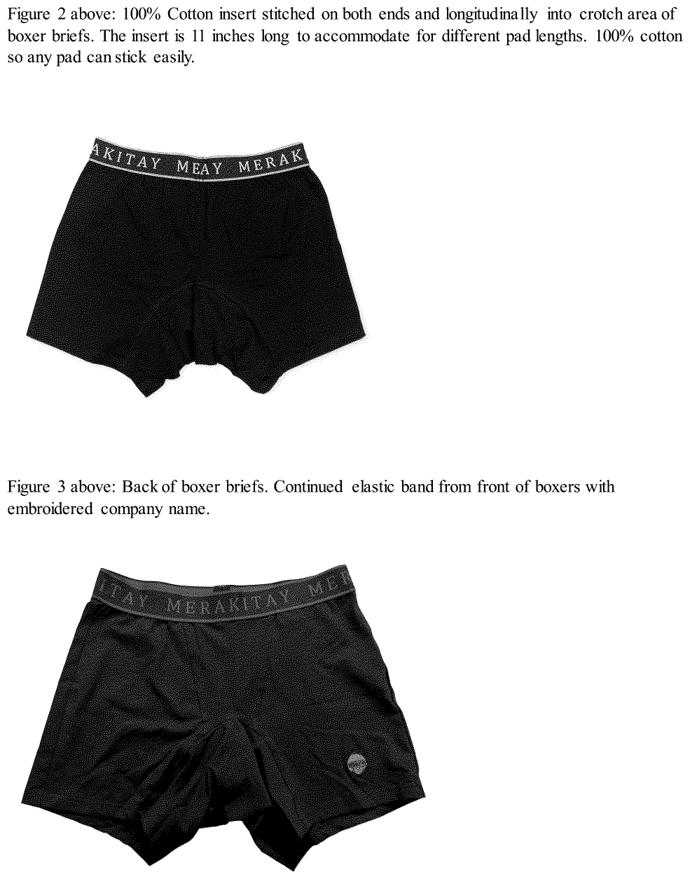

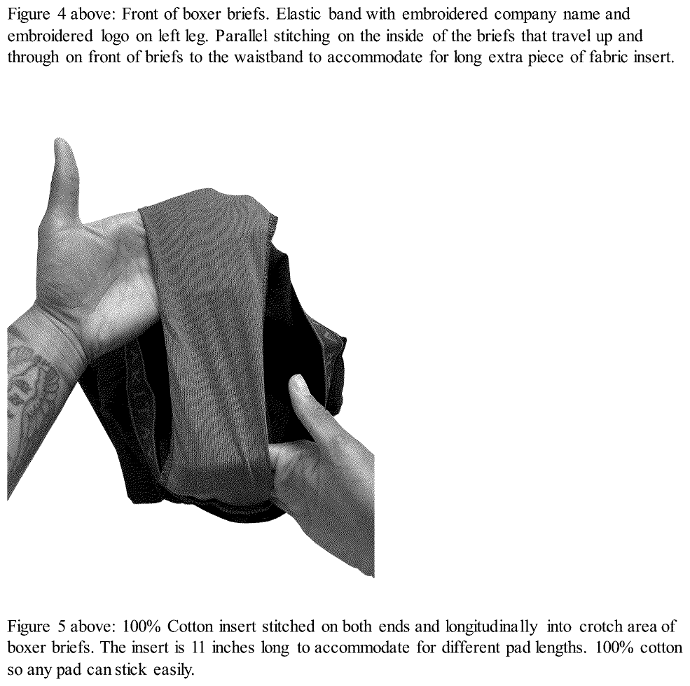

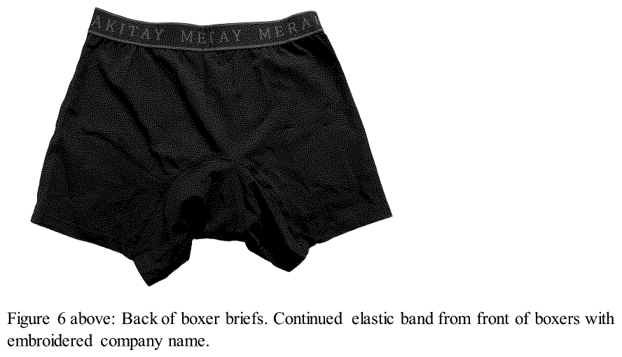

### I. BACKGROUND

Boxer briefs are made for men. The dimensions, the comfort, and the length are all made for men. Women have “boy cut” underwear, but they are much shorter and do not provide the length of men's boxer briefs. There are so many humans out there that enjoy wearing men's boxer briefs. Some of those people being women, trans men, and non-binary people. Some of those people do not feel comfortable wearing women's panties-which is the only thing you can wear with the function of adding a pad with wings or a panty liner. The modified boxer briefs with pad application not only serves the practical function of being able to wear traditional style boxer briefs during menstrual cycles, but also serves an emotional and mental function of comfort for those that feel better wearing boxer briefs.

### II. SUMMARY

Boxer brief style underwear modified with an extra piece of fabric stitched in the crotch area, 11 inches long, for menstrual cycles. This extra piece of fabric is 100% cotton with the ability to apply pad products with wings or panty liners. The boxer briefs themselves are made of 90% cotton, 5% spandex, and 5% polyester making the material moisture wicking. The boxer briefs for pad application provide comfort and practicality to those who feel better in boxer briefs over panties during menstrual cycles.

### III. DESCRIPTION OF DRAWINGS

### IV. DETAILED DESCRIPTION OF THE INVENTION

The modified boxer briefs are traditional styled boxer briefs with an extra piece of fabric stitched into the crotch area for pad application due to menstrual cycles. The boxer briefs have a 4.5-inch inseam as of right now. Inseams will change as inventory expands to help the boxers fit on more body types. Such as 4.5-inch inseam, 5.5-inch inseam, 6.5-inch inseam, and 7.5-inch inseams. The boxer briefs are made of 90% cotton, 5% polyester, and 5% spandex. This allows the fabric to be moisture wicking, accounting for sweat. A stretchy elastic waistband with the company name embroidered is placed at the top of the boxer briefs. An extra piece of 100% cotton fabric that is 11 inches long is stitched on both ends of the fabric to fit longitudinally in the crotch area of the boxer briefs for pad application due to menstrual cycles. There is parallel stitching on the front of the boxers to add more support for the extra piece of cotton insert. This helps secure pads placed on the extra piece of 100% cotton insert.

To use the modified boxer briefs is a lot like using normal boxer briefs. The only difference is that during menstrual cycles, a pad with wings or a panty liner can be placed on the 100% piece of fabric insert. Due to the fitting of the briefs and the extra insert being stitched directly in the crotch area, the pad is held securely in place. Changing of menstrual products are the same as usual.

1. A method of utilizing boxer briefs for menstrual cycles comprising:
i. An extra piece of 100% cotton fabric stitched longitudinally on both ends of said fabric of the crotch area, and
ii. Parallel stitching accompanied on inside of the front of boxer briefs to assist in the stability of said extra piece of fabric,
Whereby said piece of fabric will be used for menstrual products application for those who experience a menstrual period and enjoy wearing boxer briefs instead of panties or other underwear advertised for menstrual cycles.
